(19) United States
(12) Patent Application Publication
(10) Pub. No.: US 2025/0108238 A1
(43) Pub. Date: Apr. 3, 2025

(54) ULTRASOUND GENERATION DEVICE

(71) Applicant: JEISYS MEDICAL INC., Seoul (KR)

(72) Inventors: Hye Jin Sun, Seoul (KR); Dong Hwan Kang, Seoul (KR)

(73) Assignee: JEISYS MEDICAL INC., Seoul (KR)

(21) Appl. No.: 18/978,299

(22) Filed: Dec. 12, 2024

Related U.S. Application Data
(63) Continuation of application No. PCT/KR2023/008199, filed on Jun. 14, 2023.

(30) Foreign Application Priority Data
Jun. 14, 2022 (KR) 10-2022-0072247
Jun. 12, 2023 (KR) 10-2023-0074950

(51) Int. Cl. A61N 7/02; A61N 7/00

(52) U.S. Cl. CPC A61N 7/02; A61N 2007/0008

(57) ABSTRACT

An ultrasound generation device includes: an ultrasound generator for irradiating the skin with ultrasound energy; and a controller for controlling the operation of the ultrasound generator. The controller controls the ultrasound generator so that the ultrasound generator applies a specific ultrasound energy corresponding to at least one of fat increase, fat reduction, of elastic fiber increase in a skin layer.

### CROSS-REFERENCE TO RELATED APPLICATIONS

The present application is a continuation of International Patent Application No. PCT/KR2023/008199, filed on Jun. 14, 2023, which claims the benefit of priority to Korean Patent Application Nos. 10-2022-0072247 filed on Jun. 14, 2022 and 10-2023-0074950 filed on Jun. 12, 2023. The disclosures of the above-listed applications are hereby incorporated by reference herein in their entirety.

### FIELD

The present disclosure relates to an ultrasound generation device that can selectively provide an increase or decrease in a subcutaneous fat layer, or an enhancement of elasticity, depending on the applied ultrasound energy when ultrasound energy is applied.

### BACKGROUND

Ultrasound refers to waves with a frequency of 20 kHz or higher, and has the property of penetrating water, so it is widely used in the medical field, such as ultrasound diagnostic devices and ultrasound treatment devices.

An application of ultrasound in the medical field is an ultrasound imaging device that utilizes the properties of ultrasound penetration and reflection. For example, there is a device that visualizes the time and intensity of reflection as ultrasound penetrates the human body and passes through each organ, thereby obtaining a cross-sectional image of the human body.

In addition, there is a device that uses the heat generated by high-intensity focused ultrasound (HIFU) to burn and remove specific subcutaneous tissues, such as tumors in the skin, or to induce degeneration and regeneration of skin tissue, resulting in skin beauty or skin plastic surgery effects, such as wrinkle improvement.

Currently, the ultrasound generation devices in the skin beauty market are used for lifting procedures by focusing ultrasound energy on the SMAS layer of the skin, or for fat reduction procedures by focusing strong energy on the subcutaneous fat layer to destroy fat cells.

These ultrasound generation devices can focus ultrasound at a desired depth and adjust the desired depth. They are used to deliver effective energy to a desired depth without damaging the epidermis.

There are two methods for delivering energy with the ultrasound generation device: the dot method and the linear method. The dot method is the most widely used method, and energy is applied at a stationary location while the transducer moves and stops repeatedly, and energy is applied in the form of one or more fixed points. The linear method is a method in which energy is continuously applied while the transducer moves a specific distance at a constant speed, thereby creating linear heat formation in the skin tissue.

The dot type is a classically used method and is widely used for lifting, and the linear type is effectively used for fat reduction because it allows bulk heating.

However, when applying strong ultrasonic energy in the dot type and linear type, epidermal burns can occur, and inflammatory substances can be generated, causing problems in the dermis layer, which are caused by excessive tissue coagulation.

In addition, when performing ultrasound treatment with strong energy like the conventional method in an area where there is no fat layer, the problem of further reduction in the fat layer often occurs, or ultrasound treatment is not recommended for people with thin fat layers.

Therefore, there is a need to find a range of energy and methods that not only reduce fat in a desired area with ultrasound, but also promote elasticity and increase the fat layer.

### SUMMARY

In an exemplary embodiment, the present disclosure provides an ultrasound generation device. The device comprises: an ultrasound generator configured to irradiate ultrasound to skin; and a controller configured to control an operation of the ultrasound generator. The controller is configured to control the ultrasound generator to apply specific ultrasound energy corresponding to at least one of fat increase, fat reduction, or elastic fiber increase for a skin layer.

### BRIEF DESCRIPTION OF THE FIGURES

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 depicts a diagram illustrating a configuration of an ultrasound generation device according to the present disclosure.

FIG. 2 depicts a diagram illustrating an example of a process in which an ultrasound generator irradiates ultrasound.

FIG. 3 depicts tissue images illustrating results of fat reduction and fat increase in an obese rat model using the ultrasound generation device of FIG. 1.

FIG. 4 depicts graphs illustrating results of an experiment using the ultrasound generation device of FIG. 1 in an obese rat model, with Depth1 being the 1 mm deep upper dermis, Depth2 being the 2 mm deep dermis layer, and Depth3 being the 3 mm deep fat layer.

FIG. 5 depicts a diagram illustrating results of elasticity improvement and fat reduction and fat increase in an obese rat model using the ultrasound generation device of FIG. 1.

FIGS. 6A-6B depict graphs illustrating results of inflammatory substance expression in an obese rat model using the ultrasound generation device of FIG. 1.

FIG. 7 depicts Herovici stains illustrating results of elasticity improvement in an obese rat model using the ultrasound generation device of FIG. 1.

### DETAILED DESCRIPTION

Exemplary embodiments of the present disclosure improve elasticity of a dermal layer or selectively provide an increase or decrease in a subcutaneous fat layer depending on the specific ultrasonic energy applied when ultrasonic energy is applied.

Technical problems addressed by the present disclosure are not limited to the technical problems mentioned above, and other technical problems not mentioned will be clearly understood by those skilled in the art from the following description.

In an aspect of the present disclosure, an ultrasound generation device may include an ultrasound generator configured to irradiate ultrasound to skin; and a controller configured to control an operation of the ultrasound generator, wherein the controller is configured to control the ultrasound generator to apply specific ultrasound energy corresponding to at least one of fat increase, fat reduction, and elastic fiber increase of a skin layer.

Furthermore, the ultrasound generator may be configured to generate a focus point at a vertical distance of 0.1 mm to 13 mm from a surface of the skin for at least one of the fat increase, the fat reduction, and the elastic fiber increase, and the focus point may include a dot type in which the focus point is generated according to a predetermined interval between the focus points while the focus point moves in a horizontal direction, and a linear type in which there is no distance between the focus points.

Furthermore, the controller may be configured to control the ultrasound generator to apply specific ultrasound energy of 0.1 J to 3 J in a frequency range of 2 MHz to 20 MHz.

Furthermore, the controller may be configured to control the ultrasound generator to apply ultrasound energy of 0.1 J to 0.3 J to a fat layer in the dot type of the ultrasound generator for the fat increase.

Furthermore, the controller may be configured to control the ultrasound generator to apply 0.2 J to 0.6 J of ultrasound energy to a fat layer or the fat layer from sub dermis in the dot type of the ultrasound generator for the fat reduction, or apply 0.2 J to 0.6 J of ultrasound energy to the fat layer or the sub dermis in the linear type for the fat reduction.

Furthermore, the controller may be configured to control the ultrasound generator to apply 0.2 J to 0.6 J of ultrasound energy to a dermis layer in the linear type of the ultrasound generator for the elastic fiber increase.

Furthermore, the controller may be configured to control the ultrasound generator to apply 0.1 J to 0.3 J of ultrasound energy to a fat layer in the dot type of the ultrasound generator, and apply 0.2 J to 0.6 J of ultrasound energy to a dermis layer in the linear type of the ultrasound generator for the fat increase and the elastic fiber increase.

Furthermore, the controller may be configured to control the ultrasound generator to apply 0.1 J to 0.6 J of ultrasound energy to a fat layer or the fat layer from sub dermis in the linear type of the ultrasound generator when the ultrasound generator moves to a specific point, and apply 0.2 J to 0.6 J of ultrasound energy to a dermis layer in the dot type of the ultrasound generator when the ultrasound generator moves back to an origin, or apply 0.1 J to 0.6 J of ultrasound energy to the fat layer or the fat layer from sub dermis in the dot type of the ultrasound generator when the ultrasound generator moves to a specific point, and apply 0.2 J to 0.6 J of ultrasound energy to a dermis layer in the linear type of the ultrasound generator when the ultrasound generator moves back to the origin, for the fat reduction and the elastic fiber increase.

Furthermore, the ultrasound generator may be configured to irradiate the ultrasound to 0.1 mm to 13 mm of the skin layer and a fat layer with a frequency band of 2 MHz, 4 MHz, 5.5 MHz, 7 MHz, 10 MHz, and 20 MHz.

In the drawings, the same reference numeral refers to the same element. This disclosure does not describe all elements of embodiments, and general contents in the technical field to which the present disclosure belongs or repeated contents of the embodiments will be omitted. The terms, such as “unit, module, member, and block” may be embodied as hardware or software, and a plurality of “units, modules, members, and blocks” may be implemented as one element, or a unit, a module, a member, or a block may include a plurality of elements.

Throughout this specification, when a part is referred to as being “connected” to another part, this includes “direct connection” and “indirect connection”, and the indirect connection may include connection via a wireless communication network. Furthermore, when a certain part “includes” a certain element, other elements are not excluded unless explicitly described otherwise, and other elements may in fact be included.

Furthermore, when a certain part “includes” a certain element, other elements are not excluded unless explicitly described otherwise, and other elements may in fact be included.

In the entire specification of the present disclosure, when any member is located “on” another member, this includes a case in which still another member is present between both members as well as a case in which one member is in contact with another member.

The terms “first,” “second,” and the like are just to distinguish an element from any other element, and elements are not limited by the terms.

The singular form of the elements may be understood into the plural form unless otherwise specifically stated in the context.

Identification codes in each operation are used not for describing the order of the operations but for convenience of description, and the operations may be implemented differently from the order described unless there is a specific order explicitly described in the context.

Hereinafter, operation principles and embodiments of the present disclosure will be described with reference to the accompanying drawings.

High Intensity Focused Ultrasound (HIFU) technology is the latest thermal ablation treatment that burns specific subcutaneous tissues such as tumors in the skin by using the heat generated when high-intensity ultrasound is focused on one point in the skin. This is similar to the principle of focusing warm sunlight with a magnifying glass to light a fire. Since ultrasound easily passes through body tissues, HIFU treatment is performed in a completely non-invasive manner without a knife or even a needle. In other words, by simply pressing the patient's skin on the ultrasound generation surface, specific subcutaneous tissues such as tumors are burned and treated. In addition, HIFU treatment is currently being used to treat uterine fibroids, bone metastases, prostate cancer, breast cancer, pancreatic cancer, liver cancer, and kidney cancer.

This high-intensity focused ultrasound technology may be implemented through an ultrasound generation device. An ultrasound generation device can irradiate ultrasound to the surface of a patient's skin.

The controller of the ultrasound generation device according to the present disclosure in this specification includes various devices that may perform computational processing and provide results to a user. For example, the controller of the ultrasound generation device according to the present disclosure may include a computer, a server device, and a portable terminal, or may be in the form of one of them.

Here, the computer may include, for example, a notebook, a desktop, a laptop, a tablet PC, a slate PC, and the like mounted with a web browser.

The server device is a server that communicates with an external device to process information, and may include an application server, a computing server, a database server, a file server, a mail server, a proxy server, and a web server.

A portable terminal is a wireless communication device that ensures portability and mobility, and may include all kinds of handheld-based wireless communication devices such as PCS (Personal Communication System), GSM (Global System for Mobile communications), PDC (Personal Digital Cellular), PHS (Personal Handyphone System), PDA (Personal Digital Assistant), IMT (International Mobile Telecommunication)-2000, CDMA (Code Division Multiple Access)-2000, W-CDMA (W-Code Division Multiple Access), WiBro (Wireless Broadband Internet) terminal, a smart phone, and the like, and a wearable device such as at least one of a watch, a ring, bracelets, anklets, a necklace, glasses, contact lenses, or a head-mounted device (HMD).

The ultrasound generation device according to the present disclosure may control an ultrasound generator to apply specific ultrasound energy corresponding to at least one of fat increase, fat decrease, and elastic fiber increase of the skin layer or fat layer.

The ultrasound generation device may selectively provide an effect of increasing or decreasing the subcutaneous fat layer or improving the elasticity of the skin layer according to the specific ultrasound energy applied when ultrasound energy is applied.

Hereinafter, an example embodiment of the ultrasound generation device will be described in detail.

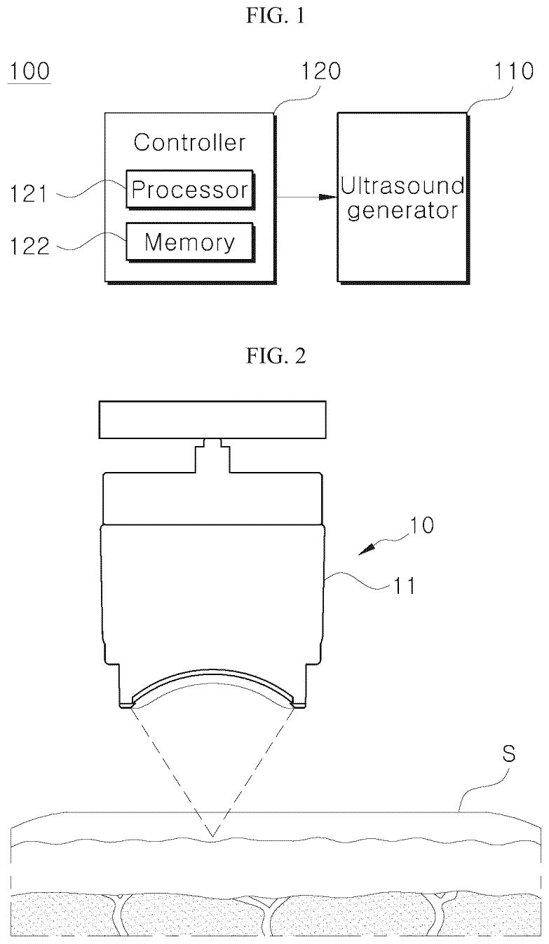

FIG. 1 depicts a diagram illustrating a configuration of an ultrasound generation device according to the present disclosure. FIG. depicts is a diagram illustrating an example of a process in which an ultrasound generator irradiates ultrasound.

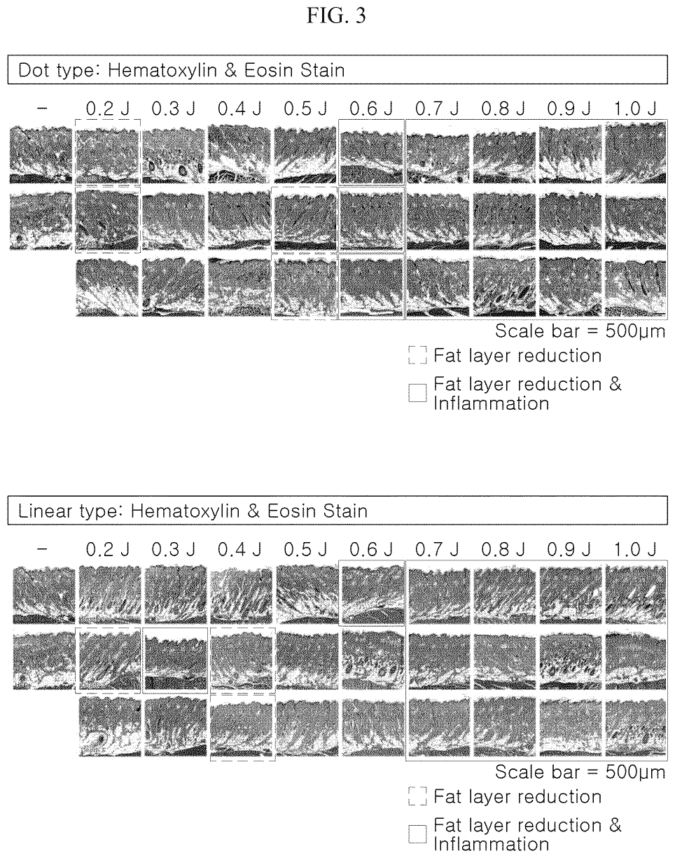

Referring to FIGS. 1 and 2, an ultrasound generation device 100 may include an ultrasound generator 110 and a controller 120.

The ultrasound generator 110 having a transducer 11 may irradiate ultrasound to a skin S while maintaining the ultrasound focus depth.

The controller 120 may be implemented with a memory 122 that stores data on an algorithm for controlling the operation of components within the device or a program that reproduces the algorithm, and at least one processor 121 that performs the operation using the data stored in the memory 122. Here, the memory 122 and the processor 121 may be implemented as separate chips, respectively. In addition, the memory 122 and the processor 121 may be implemented as a single chip.

The memory 122 may store data supporting various functions of the device, programs for the operation of the control unit, may store input/output data, a plurality of application programs (or applications) executed on the device, data for the operation of the device, and commands. At least some of these application programs may be downloaded from an external server via wireless communication.

The memory 122 may include at least one type of storage medium among a flash memory type, a hard disk type, an SSD type (Solid State Disk type), an SDD type (Silicon Disk Drive type), a multimedia card micro type, a card type memory (for example, an SD or XD memory, etc.), a random access memory (RAM), a static random access memory (SRAM), a read-only memory (ROM), an electrically erasable programmable read-only memory (EEPROM), a programmable read-only memory (PROM), a magnetic memory, a magnetic disk, or an optical disk. In addition, the memory 122 may be a database that is separate from the device but connected by wire or wirelessly.

The processor 121 may control the ultrasound generator 110 so that the ultrasound generator 110 applies specific ultrasound energy corresponding to at least one of fat increase, fat reduction, or elastic fiber increase of the skin layer. At this time, the processor 121 may control the ultrasound generator 110 so that the ultrasound generator 110 applies specific ultrasound energy corresponding to at least one of improving elasticity of the skin layer or fat increase or fat reduction of the subcutaneous fat layer.

The ultrasound generator 110 may generate a focus point at a vertical distance of 0.1 mm to 13 mm from a skin surface for at least one of the fat increase, the fat reduction, or the elastic fiber (collagen, elastin fiber) increase. At this time, the ultrasound generator 110 may include a dot type in which a focus point is generated according to a predetermined interval between focus points while the focus point moves in a horizontal direction, and a linear type in which there is no distance between focus points.

The processor 121 may control the ultrasound generator 110 to apply specific ultrasound energy of 0.1 J to 3 J in a frequency range of 2 MHz to 20 MHz. For example, the ultrasound generator 110 may irradiate ultrasound to 0.1 mm to 13 mm of the skin layer and the fat layer in a frequency band of 2 MHz, 4 MHz, 5.5 MHz, 7 MHz, 10 MHz, and 20 MHz.

The processor 121 may control the ultrasound generator 110 to apply ultrasound energy of 0.1 J to 0.3 J to the fat layer in the dot type of the ultrasound generator 110 for the fat increase. For example, the doctor may control the ultrasound generator 110 to check a depth so that the ultrasound energy is focused on the fat layer of the subject's examination area to a depth of 0.1 mm to 13 mm of the skin, and the processor 121 may control the ultrasound generator 110 to apply 0.2 J of ultrasound energy to the fat layer in the dot type of 2 MHz to 7 MHz.

The processor 121 may control the ultrasound generator 110 to apply 0.2 J to 0.6 J of ultrasound energy to the fat layer or the sub dermis in the dot type of the ultrasound generator 110 or to apply 0.2 J to 0.6 J of ultrasound energy to the fat layer or the sub-dermis in the linear type for fat reduction. For example, the doctor may check the depth to focus on the fat layer or the sub dermis of the subject's examination area to a depth of 0.1 mm to 13 mm of the skin, and the processor 121 may control the ultrasound generator 110 to apply 0.2 J to 0.6 J of ultrasound energy to the fat layer or the sub dermis of the subject's examination area in the dot type or the linear type of 2 MHz to 7 MHz.

The processor 121 may control the ultrasound generator 110 to apply 0.2 J to 0.6 J of ultrasound energy to the dermis lay in the linear type of the ultrasound generator 110 for the elastic fiber increase. For example, the doctor may control the ultrasound generator 110 to check the depth so that the ultrasound energy is focused on the dermis layer of the subject's examination area to a depth of 0.1 mm to 13 mm of the skin, and the processor 121 may control the ultrasound generator 110 to apply 0.2 J to 0.6 J of ultrasound energy to the dermis layer in the linear type of 2 MHz to 20 MHz of the ultrasound generator 110.

The processor 121 may control the ultrasound generator 110 to apply 0.1 J to 0.3 J of ultrasound energy to the fat layer in the dot type of the ultrasound generator 110 and to apply 0.2 J to 0.6 J of ultrasound energy to the dermis layer in the linear type of the ultrasound generator 110 for the fat increase and the elastic fiber increase. For example, the doctor may control the ultrasound generator 110 to check the depth so that the ultrasound energy is focused on the fat layer of the subject's examination area to a depth of 0.1 mm to 13 mm of the skin, and the processor 121 may control the ultrasound generator 110 to apply 0.1 J to 0.3 J of ultrasound energy to the fat layer in the dot type of 2 MHz to 20 MHz of the ultrasound generator 110. In addition, the doctor may control the ultrasound generator 110 to check the depth so that the ultrasound energy is focused on the dermis layer of the subject's examination area to a depth of 0.1 mm to 13 mm of the skin, and the processor 121 may control the ultrasound generator 110 to apply 0.2 J to 0.6 J of ultrasound energy to the dermis layer in the linear type of 2 MHz to 20 MHz of the ultrasound generator 110.

The processor 121 may control the ultrasound generator 110 to apply 0.1 J to 0.6 J of ultrasound energy to the fat layer or the sub dermis in the linear type of the ultrasound generator 110 when the ultrasound generator 110 moves to a specific point for the fat reduction and the elastic fiber increase, and to apply 0.2 J to 0.6 J of ultrasound energy to the dermis layer in the dot type of the ultrasound generator 110 when the ultrasound generator 110 moves back to the origin. For example, the doctor may control the ultrasound generator 110 to check the depth so that the ultrasound energy is focused on the fat layer or the sub dermis of the subject's examination area to a depth of 0.1 mm to 13 mm of the skin, and the processor 121 to apply 0.1 J to 0.6 J of ultrasound energy to the fat layer or the sub dermis of the subject's examination area in the linear type of 2 MHz to 20 MHz of the ultrasound generator 110 when the ultrasound generator 110 moves to a specific point, and to apply 0.2 J to 0.6 J of ultrasound energy to the dermis layer in the dot type of 2 MHz to 20 MHz of the ultrasound generator 110 when the ultrasound generator 110 moves back to the origin.

On the other hand, the processor 121 may control the ultrasound generator 110 to apply 0.1 J to 0.6 J of ultrasound energy to the fat layer or the sub dermis in the dot type of the ultrasound generator 110 when the ultrasound generator 110 moves to a specific point for the fat reduction and the elastic fiber increase, and to apply 0.2 J to 0.6 J of ultrasound energy to the dermis layer in the linear type of the ultrasound generator 110 when the ultrasound generator 110 moves back to the origin. For example, the doctor may control the ultrasound generator 110 to check the depth so that the ultrasound energy is focused on the fat layer or the sub dermis of the subject's examination area at a depth of 0.1 mm to 13 mm of the skin, and the processor 121 to apply 0.1 J to 0.6 J of ultrasound energy to the fat layer or the sub dermis of the subject's examination area in the dot type of 2 MHz to 20 MHz of the ultrasound generator 110 when the ultrasound generator 110 moves to a specific point, and to apply 0.2 J to 0.6 J of ultrasound energy to the dermis layer in the linear type of 2 MHz to 20 MHz of the ultrasound generator 110 when the ultrasound generator 110 moves back to the origin.

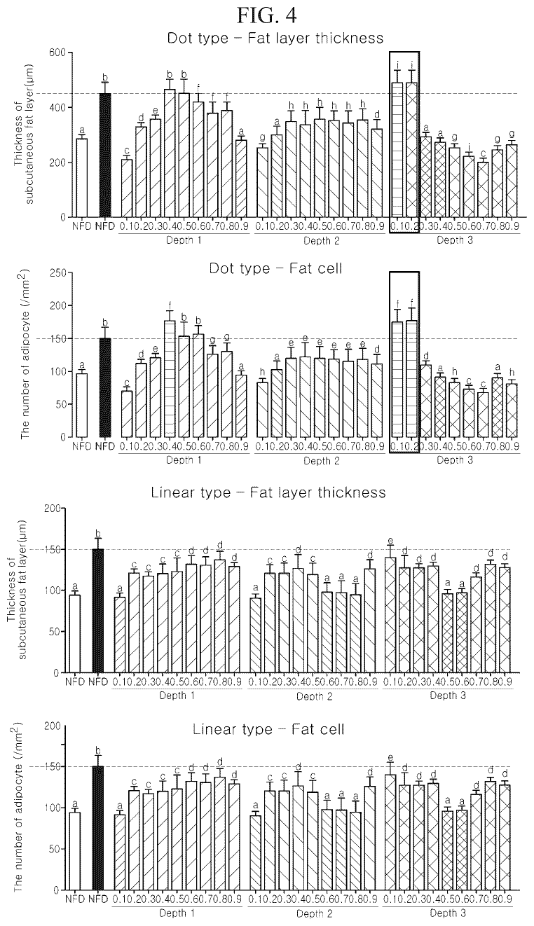

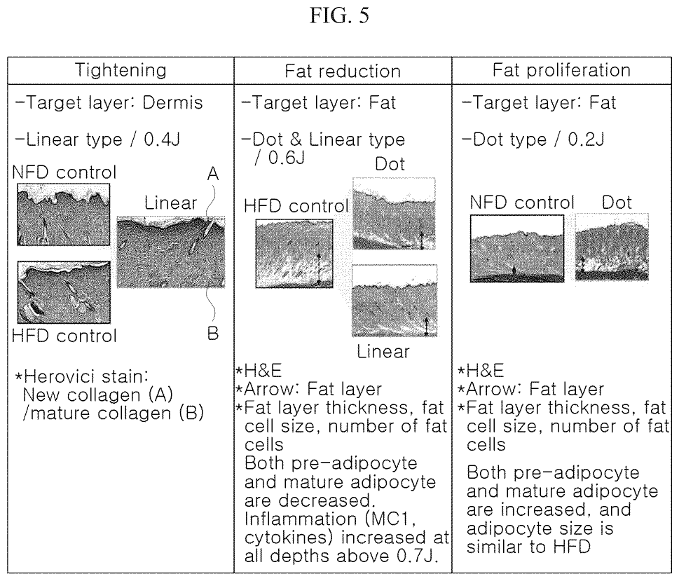

FIG. 3 depicts tissue photographs illustrating results of fat reduction and fat increase in an obese rat model using the ultrasound generation device of FIG. 1. FIG. 4 depicts graphs illustrating results of an experiment using the ultrasound generation device of FIG. 1 in an obese rat model, with Depth1 being the 1 mm deep upper dermis, Depth2 being the 2 mm deep dermis layer, and Depth3 being the 3 mm deep fat layer.

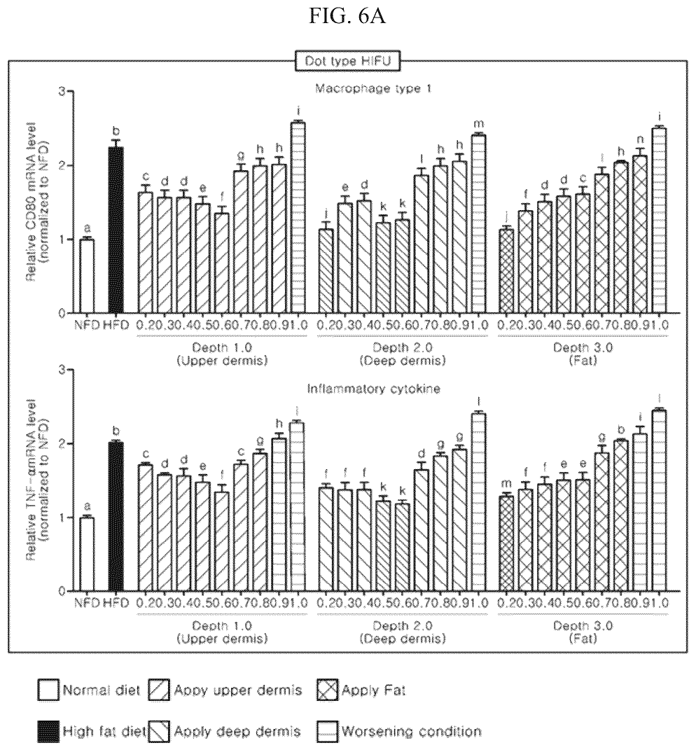

FIG. 5 depicts a diagram illustrating results of elasticity improvement and fat reduction and fat increase in an obese rat model using the ultrasound generation device of FIG. 1.

When the dot type and the linear type of the ultrasound generator of FIG. 3 are applied at the same depth and with the same ultrasound energy, the tissue photograph illustrating the change in fat tissue are observed, and FIG. 4 quantifies the tissue result of FIG. 3 and shows the result as a graph. As a result of the tissue experiment in the obese rat model, when applying 0.2 J of ultrasound energy in the dot type to the fat layer and comparing it with the other conditions of FIG. 3, it is identified that it is effective in the fat increase. And, in the fat layer or the sub dermis, both the dot type and the linear type of the ultrasound generator of FIG. 3 applied 0.2 J to 1.0 J of ultrasound energy to the fat layer, and the fat reduction occurred, but from 0.7 J, inflammatory substances are expressed, and it is identified that the most effective range is 0.2 J to 0.6 J by excluding these.

As shown in FIG. 5, in the tissue photographs, it is identified that the fat layer increased or decreased when ultrasound energy is applied with the dot type of the ultrasound generator of FIG. 3 and the linear type of the ultrasound generator of FIG. 4.

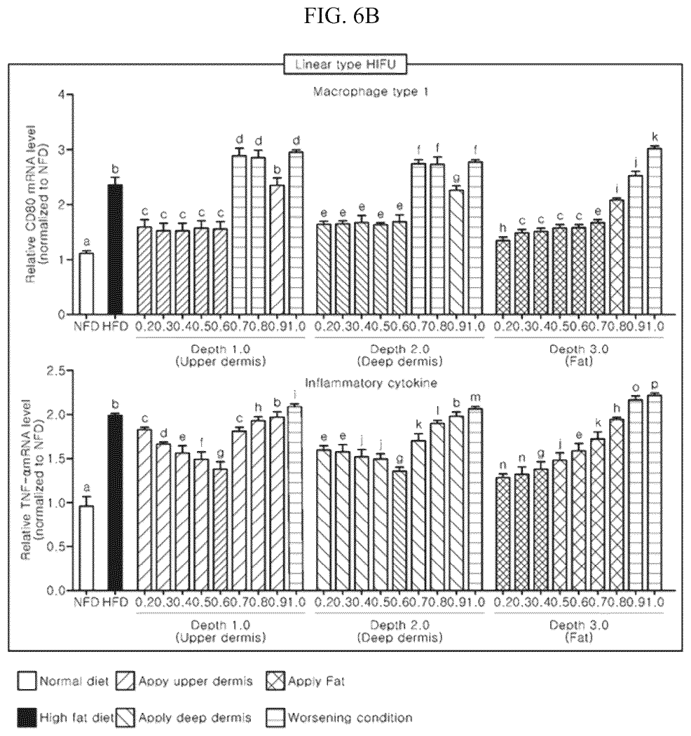

FIGS. 6A-6B depict graphs illustrating results of inflammatory substance expression in an obese rat model using the ultrasound generation device of FIG. 1.

FIGS. 6A-6B depict graphs of inflammation (Macrophage 1, inflammatory cytokine) level, and it is identified that the inflammation levels decreased from 0.2 J to 0.6 J in both the dot type of the ultrasound generator of FIG. 6A and the linear type of the ultrasound generator of FIG. 6B, and increased from 0.7 J or higher.

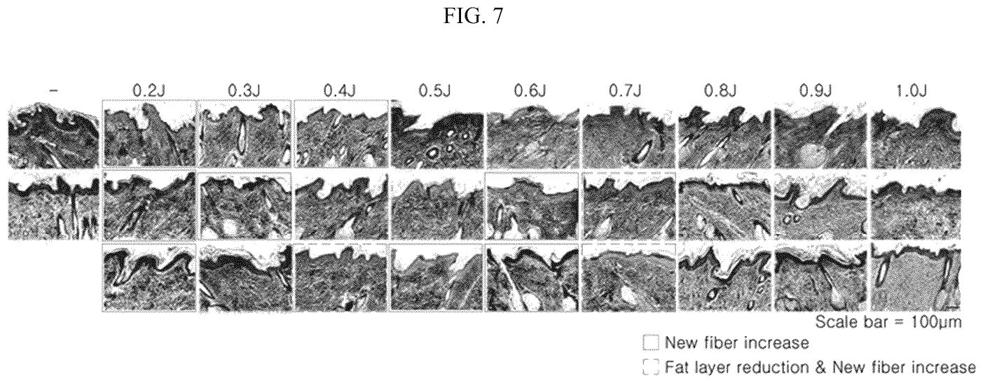

FIG. 7 depicts Herovici stains illustrating results of elasticity improvement in an obese rat model using the ultrasound generation device of FIG. 1.

FIG. 7 depicts Herovici stains, and it is identified that the increase in new collagen and elastin fibers is identified, and it is identified that the increase is the greatest at 0.2 J to 0.6 J in the linear type.

Therefore, the ultrasound generation device according to the present disclosure may selectively provide an effect of increasing or decreasing a subcutaneous fat layer or improving the elasticity of a skin layer depending on the specific ultrasound energy applied and the skin layer applied when ultrasound energy is applied.

Meanwhile, the depth of the dermis layer and the fat layer are different depending on the person or the part, and the skin structure may be identified by measuring the relevant part with a skin measuring device, and a cartridge select one from 0.1 mm to 13 mm of a depth appropriate therefor may be selected.

At least one component may be added or deleted in accordance with the performance of the components illustrated in FIG. 1. In addition, it will be easily understood by those skilled in the art that the mutual positions of the components may be changed in accordance with the performance or structure of the system.

As described above, the disclosed embodiments have been described with reference to the attached drawings. A person skilled in the art will appreciate that the present disclosure may be practiced in other forms than the disclosed embodiments without changing the technical spirit or essential features of the present disclosure. The disclosed embodiments are illustrative and should not be construed as limiting.

According to exemplary embodiments of the present disclosure, when ultrasonic energy is applied, an effect is provided that can selectively improve the elasticity of the dermal layer or provide an increase or decrease in the subcutaneous fat layer depending on the specific ultrasonic energy applied.

The effects of the present disclosure are not limited to the effects mentioned above, and other effects that are not mentioned will be clearly understood by those skilled in the art from the description.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. An ultrasound generation device, comprising:
an ultrasound generator configured to irradiate ultrasound to skin; and
a controller configured to control an operation of the ultrasound generator;
wherein the controller is configured to control the ultrasound generator to apply specific ultrasound energy corresponding to at least one of fat increase, fat reduction, or elastic fiber increase for a skin layer.

2. The device according to claim 1, wherein the ultrasound generator is configured to generate a focus point at a vertical distance of 0.1 mm to 13 mm from a surface of the skin for at least one of fat increase, fat reduction, or elastic fiber increase.

3. The device according to claim 2, wherein the specific ultrasound energy is in a range of 0.1 J to 3 J and has a frequency in a frequency range of 2 MHz to 20 MHz.

4. The device according to claim 1, wherein the controller is configured to control the ultrasound generator to operate as a dot type ultrasound generator which focuses the irradiated ultrasound at focus points according to a predetermined interval while the ultrasound generator moves horizontally.

5. The device according to claim 4, wherein the specific ultrasound energy is in a range of 0.1 J to 0.3 J and is applied to a fat layer for fat increase.

6. The device according to claim 4, wherein the specific ultrasound energy is in a range of 0.2 J to 0.6 J and is applied to a fat layer for fat reduction; or
wherein the specific ultrasound energy is in a range of 0.2 J to 0.6 J and is applied to a fat layer via application to a sub dermis layer for fat reduction.

7. The device according to claim 4, wherein the specific ultrasound energy is in a range of 0.2 J to 0.6 J and is applied to a dermis layer for elastic fiber increase.

8. The device according to claim 1, wherein the controller is configured to control the ultrasound generator to operate as a linear type ultrasound generator which continuously focuses the irradiated ultrasound at different positions corresponding to movement of the ultrasound generator.

9. The device according to claim 8, wherein the specific ultrasound energy is in a range of 0.2 J to 0.6 J of ultrasound energy and is applied to a fat layer for fat reduction; or
wherein the specific ultrasound energy is in a range of 0.2 J to 0.6 J of ultrasound energy and is applied to a fat layer via application to a sub dermis layer for fat reduction.

10. The device according to claim 8, wherein the specific ultrasound energy is in a range of 0.2 J to 0.6 J and is applied to a dermis layer for elastic fiber increase.

11. The device according to claim 1, wherein the controller is configured to:
control the ultrasound generator to operate as a dot type ultrasound generator and apply specific ultrasound energy in a range of 0.1 to 0.3 J to a fat layer for fat increase; and
control the ultrasound generator to operate as a linear type ultrasound generator and apply specific ultrasound energy in a range of 0.2-0.6 J to a dermis layer for elastic fiber increase.

12. The device according to claim 1, wherein the controller is configured to control the ultrasound generator to operate as a linear type ultrasound generator during a first movement and as a dot type ultrasound generator during a second movement.

13. The device according to claim 12, wherein the first movement is a movement from a first position to a second position and the second movement is a movement from the second position to the first position.

14. The device according to claim 13, wherein the controller is configured to control the ultrasound generator to apply 0.1 J to 0.6 J of ultrasound energy to a fat layer during the first movement and to apply 0.2 J to 0.6 J of ultrasound energy to a dermis layer during the second movement.

15. The device according to claim 14, wherein the application of the ultrasound energy during the first and second movements is for fat reduction and elastic fiber increase.

16. The device according to claim 1, wherein the controller is configured to control the ultrasound generator to operate as a dot type ultrasound generator during a first movement and as a linear type ultrasound generator during a second movement.

17. The device according to claim 16, wherein the first movement is a movement from a first position to a second position and the second movement is a movement from the second position to the first position.

18. The device according to claim 17, wherein the controller is configured to control the ultrasound generator to apply 0.1 J to 0.6 J of ultrasound energy to a fat layer during the first movement and to apply 0.2 J to 0.6 J of ultrasound energy to a dermis layer during the second movement.

19. The device according to claim 18, wherein the application of the ultrasound energy during the first and second movements is for fat reduction and elastic fiber increase.

20. The device according to claim 1, wherein the ultrasound generator is configured to irradiate the ultrasound to a depth of 0.1 mm to 13 mm and with a frequency of 2 MHz, 4 MHz, 5.5 MHz, 7 MHz, 10 MHz, and 20 MHz.
